(19) United States
(12) Patent Application Publication
(10) Pub. No.: US 2026/0351642 A1
(43) Pub. Date: Nov. 5, 2026

(54) NOVEL COVALENT ORGANIC FRAMEWORK, ELECTRODE COMPOSITION, MEMBRANE-ELECTRODE ASSEMBLY, AND FUEL CELL

(71) Applicant: KOREA INSTITUTE OF SCIENCE AND TECHNOLOGY, Seoul (KR)

(72) Inventors: Jin Young KIM, Seoul (KR); Yeong-Seop LEE, Seoul (KR); Kyeongmin KIM, Seoul (KR); Yoon Hee JANG, Seoul (KR); Hae Jung Son, Seoul (KR)

(21) Appl. No.: 19/647,411

(22) Filed: Apr. 14, 2026

(30) Foreign Application Priority Data
Apr. 18, 2025 (KR) 10-2025-0051017

(51) Int. Cl. H01M 8/1004; C07D 471/22; H01M 4/86; H01M 4/92; H01M 8/10

(52) U.S. Cl. CPC H01M 8/1004; C07D 471/22; H01M 4/8657; H01M 4/8668; H01M 4/92; H01M 2008/1095

(57) ABSTRACT

The disclosure relates to a novel covalent organic framework (COF), an electrode composition, a membrane-electrode assembly (MEA), and a fuel cell. The disclosure provides a novel covalent organic framework (COF) having a site for coordination bonding with a catalyst, and an electrode composition, a membrane-electrode assembly (MEA), and a fuel cell including the same.

### CROSS-REFERENCE TO RELATED APPLICATION

This application claims the benefit of Korean Patent Application No. 10-2025-0051017, filed on Apr. 18, 2025, in the Korean Intellectual Property Office (now the Ministry of Intellectual Property (MOIP)), the entire disclosure of which is incorporated herein by reference for all purposes.

### BACKGROUND

### 1. Field of the Invention

One or more embodiments relate to a novel covalent organic framework (COF), an electrode composition, a membrane-electrode assembly (MEA), and a fuel cell.

### 2. Description of the Related Art

A fuel cell is an environmentally friendly energy device having high energy efficiency and has attracted attention as a next-generation power source. A polymer electrolyte membrane fuel cell (PEMFC), which is a low-temperature fuel cell, has high utility as a mobile fuel cell for automobiles and drones. PEMFC development has achieved a high level of technology in terms of low-temperature operation and energy efficiency, but improvement in cost and durability is required. Accordingly, a core material development strategy that enables long-term operation and cost reduction of PEMFC is required.

Among the components of a PEMFC, an MEA is a core material that determines the performance of a fuel cell. The MEA includes a catalyst layer where an electrode reaction occurs and an electrolyte membrane.

A main cause of performance degradation exhibited by MEA materials is deterioration in physical durability due to continuous expansion and contraction in a PEMFC driving environment.

As an ionomer for an electrode layer of a polymer electrolyte fuel cell, a commercial perfluorinated ionomer material, perfluorosulfonic acid (PFSA) ionomer (e.g., Nafion, 3M, or Aquivion), is used. The PFSA ionomer is widely used as a binder for electrodes and as an electrolyte membrane. However, since a fluorine-based polymer based on polytetrafluoroethylene (PTFE) is composed of carbon-carbon bonds or carbon-fluorine bonds having very strong bonding forces, the fluorine-based polymer has crystallinity or semi-crystallinity, which exhibits low permeability to gases or vapors. That is, commercial perfluorinated ionomer materials have excellent ion conductivity and chemical resistance, but their utility is decreasing due to problems of high cost and low gas permeability.

Therefore, research related to the improvement of ionomer gas permeability is required to facilitate gas permeation in an electrode layer, which significantly affects fuel cell and stack performance.

### SUMMARY

The disclosure provides a covalent organic framework (COF) having a site (e.g., a bridging group) that forms a coordination bond with a catalyst (e.g., a metal catalyst) and an ionic group.

The disclosure provides an electrode composition including the COF of the disclosure.

The disclosure provides an electrode for a fuel cell including the electrode composition of the disclosure.

The disclosure provides a membrane-electrode assembly (MEA) including the electrode of the disclosure. For example, the MEA may be a polymer electrolyte MEA.

The disclosure provides a fuel cell including the electrode for a fuel cell of the disclosure. For example, the fuel cell may be a polymer electrolyte membrane fuel cell (PEMFC).

The disclosure provides a method of manufacturing an electrode for a fuel cell using the electrode composition of the disclosure.

The disclosure provides a method of manufacturing an MEA using the electrode composition of the disclosure.

However, technical goals to be achieved are not limited to those described above, and other goals not mentioned above can be clearly understood by one of ordinary skill in the art from the following description.

According to an aspect, a COF of the disclosure may be a compound represented by Formula 1.

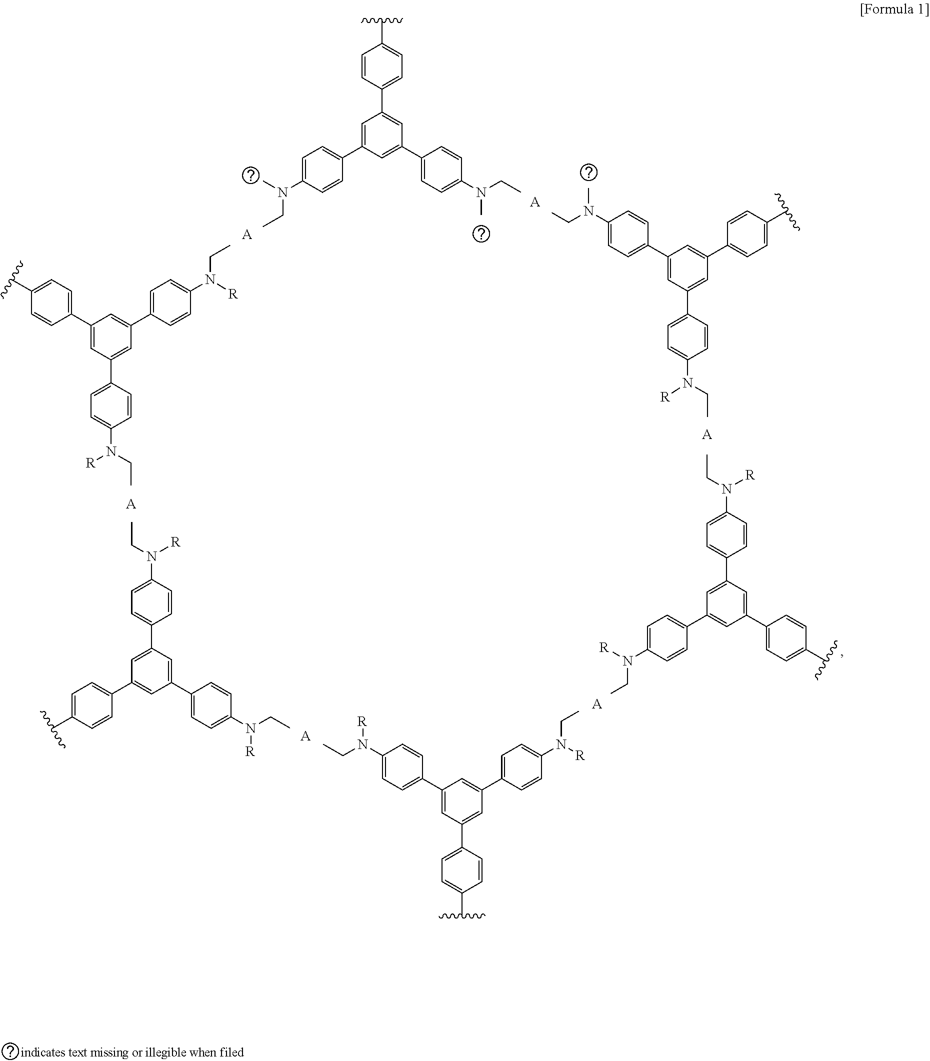

wherein R is an ionic group and is —(CH₂)n-Z⁻ n is 1 to 10, and Z⁻ is COO, SO₃⁻, or PF₅⁻, and A is a bridging group in which two or more six-membered aromatic rings, each containing one or more nitrogen atoms in a ring, are connected.

According to another aspect, an electrode composition for a fuel cell of the disclosure may include a COF that is a compound represented by Formula 1, according to embodiments of the disclosure, and a catalyst for a fuel cell.

According to still another aspect, an MEA of the disclosure may include an electrolyte membrane, and an electrode layer including the electrode composition for a fuel cell, according to embodiments of the disclosure, wherein the electrode layer is disposed on one surface or both surfaces of the electrolyte membrane.

According to yet another aspect, a fuel cell of the disclosure may include the MEA according to embodiments of the disclosure.

Additional aspects of embodiments will be set forth in part in the description which follows and, in part, will be apparent from the description, or may be learned by practice of the disclosure.

According to embodiments, the disclosure may provide a novel COF capable of forming a coordination bond with a catalyst (e.g., a metal catalyst) (e.g., a platinum catalyst) applicable to driving an MEA in a driving environment of a fuel cell (e.g., a PEMFC), enabling high oxygen permeability, and ensuring durability against the catalyst. The disclosure may also provide a fuel cell binder (e.g., a PEMFC binder) utilizing the COF. In addition, a COF-based polymer electrolyte MEA and a fuel cell utilizing the same may be provided.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1 illustrates a coordination bond between a covalent organic framework (COF-S) and a platinum catalyst in a composition including the COF, a perfluorosulfonic acid (PFSA)-based ionomer, and the platinum catalyst of the disclosure, as an example.

FIG. 2A illustrates a Fourier-transform infrared spectroscopy (FT-IR) spectrum of COF-S prepared in Example 1, according to an embodiment.

FIG. 2B illustrates an FT-IR spectrum of COF-S prepared in Example 1, according to an embodiment.

FIG. 3A shows a scanning electron microscope (SEM) photograph of COF-S capable of coordination bonding with a platinum catalyst prepared in Example 1, according to an embodiment.

FIG. 3B shows an SEM photograph of COF-S capable of coordination bonding with a platinum catalyst prepared in Example 1, according to an embodiment.

FIG. 4 illustrates an X-ray photoelectron spectroscopy (XPS) spectrum of COF-S capable of coordination bonding with a platinum catalyst synthesized in Example 1, according to an embodiment.

FIG. 5A illustrates current-voltage performance (H₂/Air) of a polymer electrolyte membrane fuel cell (PEMFC) unit cell prepared in Example 1, according to an embodiment.

FIG. 5B illustrates current-power performance (H₂/Air) of the PEMFC unit cell prepared in Example 1, according to an embodiment.

FIG. 6 illustrates an oxygen gain analysis of a PEMFC unit cell prepared in Example 1, according to an embodiment.

FIG. 7 illustrates electrochemical impedance spectroscopy (EIS) resistance measurement results of a PEMFC unit cell prepared in Example 1, according to an embodiment.

FIG. 8 illustrates resistance measured through EIS of a membrane-electrode assembly (MEA) (3M) using only a PFSA-based ionomer and a sample to which a COF-S material of Example 1 is added, according to an embodiment.

FIG. 9A illustrates oxygen permeation resistance measurement of a PEMFC unit cell prepared in Example 1, according to an embodiment.

FIG. 9B illustrates oxygen permeation resistance measurement of a PEMFC unit cell prepared in Example 1, according to an embodiment.

FIG. 9C illustrates oxygen permeation resistance measurement of a PEMFC unit cell prepared in Example 1, according to an embodiment.

FIG. 10A illustrates current-voltage performance of a PEMFC unit cell prepared in Example 1 before and after an accelerated degradation test (ADT), according to an embodiment.

FIG. 10B illustrates current-voltage performance of a PEMFC unit cell prepared in Example 1 before and after an ADT, according to an embodiment.

FIG. 10C illustrates current-voltage performance of a PEMFC unit cell prepared in Example 1 before and after an ADT, according to an embodiment.

FIG. 10D illustrates current-voltage performance of a PEMFC unit cell prepared in Example 1 before and after an ADT, according to an embodiment.

### DETAILED DESCRIPTION

Hereinafter, embodiments of the present disclosure will be described in detail with reference to the accompanying drawings. When it is determined that detailed description related to a related known function or configuration may make the purpose of the present disclosure unnecessarily ambiguous in describing the present disclosure, the detailed description will be omitted here. In addition, terminologies used herein are defined to appropriately describe the example embodiments and thus may be changed depending on a user, the intent of an operator, or a custom of a field to which the present disclosure pertains. Accordingly, the terminologies must be defined based on the following overall description of the present specification. In the drawings, like reference numerals are used for like elements.

In the whole specification, when a member is positioned “on” another member, this not only includes a case that the member is brought into contact with the other member, but also includes a case that another member exists between two members.

It will be understood that when a certain part “includes” a certain component, the part does not exclude another component but may further include another component.

As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and do not limit the components in other aspects (e.g., importance or order). For example, suitable results may be achieved if the described techniques are performed in a different order and/or if the described components are combined in a different manner and/or replaced or substituted by other components or their equivalents. Therefore, other implementations, other embodiments, and equivalents to the claims are also within the scope of the claims below.

Hereinafter, a covalent organic framework (COF) and an application thereof of the disclosure will be described in detail with reference to embodiments and drawings. However, the present disclosure is not limited to the embodiments and drawings.

According to an embodiment, the COF of the disclosure may be a compound having a site (e.g., a bridging group) coordinating with a chemical species (e.g., a metal) (e.g., a platinum catalyst) and an ionic group in a molecule. The COF of the disclosure may be a compound represented by Formula 1 below.

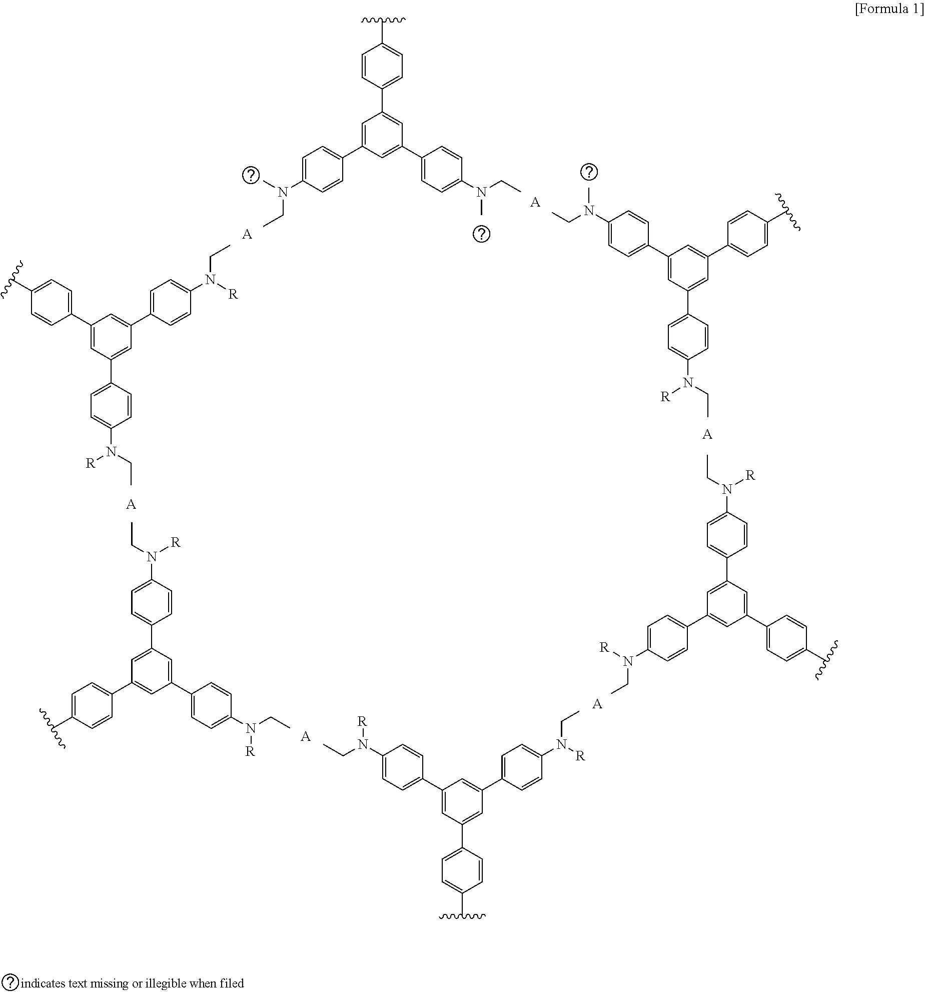

According to an embodiment, in Formula 1, R may be an ionic group and may be —(CH₂)n-Z⁻. Here, n may be an integer of 1 to 10, and Z⁻ may be COO⁻, SO₃⁻, or PF₅⁻. For example, R may be —(CH₂)n-SO₃⁻, and n may be an integer of 2 to 8; 3 to 6; or 3 to 4. In Formula 1, A may be a bridging group. The bridging group may be formed by connecting two or more six-membered aromatic rings, each containing one or more nitrogen atoms in a ring. The bridging group may include a site (e.g., a nitrogen element) that forms a coordination bond with a metal catalyst. The bridging group may be represented by

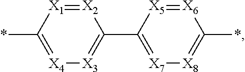

wherein at least two of X₁ to X₈ may be nitrogen, and the remainder may be carbon. (*) may denote a connection site. For example, the bridging group may be a bipyridine group in which two or more pyridines are connected. For example, the bridging group may be selected from

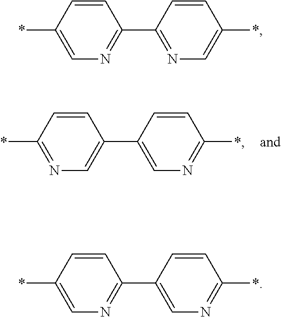

According to an embodiment, the COF may provide a site for coordination bonding with a specific chemical species and may act as a binder attaching and/or fixing the specific chemical species. For example, nitrogen in a pyridine form of the bridging group may form a coordination bond with a metal catalyst (e.g., platinum) to prevent loss and aggregation of the metal catalyst and prevent reduction in catalytic activity.

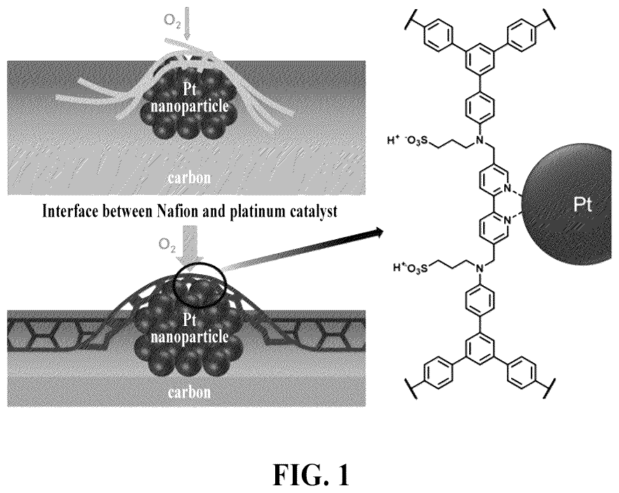

According to an embodiment, the COF may include a porous structure form. That is, in the COF, organic molecules are connected by covalent bonds to form a crystalline structure. In addition, the COF may have a high surface area and a pore volume along with a regular pore structure. The COF may be usefully utilized for gas storage, catalysts, or the like. Furthermore, the COF may improve oxygen permeability in an electrode or a membrane-electrode assembly (MEA) due to the porous structure. In addition, a structure and properties of the COF may be variously designed by modifying a type, a structure, a substituent, or the like of a monomer introduced during the manufacture of the COF. More specifically, according to an embodiment, FIG. 1 illustrates a coordination bond between a COF (e.g., a COF-S having a sulfonic acid group as an ionic group in Formula 1) and a platinum catalyst in a composition including the COF (e.g., COF-S), a perfluorosulfonic acid (PFSA)-based ionomer (e.g., Nafion), and the platinum catalyst of the disclosure, as an example. In FIG. 1, the COF represented by Formula 1 is an organic material having a porous structure form, may partially replace the use of a PFSA-based ionomer used in a fuel cell, and may increase the performance of the fuel cell by lowering the oxygen permeation resistance of an MEA. That is, in the case of a perfluorinated ionomer such as Nafion, oxygen permeation occurs less because the space for oxygen to permeate is small. However, when the COF (e.g., COF-S) represented by Formula 1 of the disclosure is used, high oxygen permeation may be implemented due to the porous structure of the COF. Furthermore, in the fuel cell, elution and aggregation of the platinum catalyst occur during an operating environment, which may cause a reduction in catalytic activity. However, in the COF represented by Formula 1, nitrogen of the bridging group (e.g., two nitrogens in pyridine) forms a coordination bond with the platinum catalyst, thereby preventing loss and aggregation of the platinum catalyst and preventing reduction in catalytic activity.

According to an embodiment, the COF may be a porous particle, and for example, may be a particle having a size of 100 nanometers (nm) to 100 micrometers (μm); 200 nm to 100 μm; 500 nm to 100 μm; 1000 nm to 100 μm; 10 μm to 100 μm; or 50 μm to 100 μm. The size may be a diameter, a width, a thickness, or a length. The porous particle may have a form such as a powder, a fiber, a sphere, a tube, a two-dimensional planar shape, or a plate shape.

According to an embodiment, the disclosure may provide an electrode composition including a COF, which is the compound represented by Formula 1 of the disclosure, and a catalyst. The electrode composition may be used for the manufacture of an electrode or a membrane electrode (layer). For example, the electrode composition may be used for the manufacture of a membrane electrode (layer) for a polymer electrolyte fuel cell. The COF may be present in an amount of 0.01 weight percent (wt %) to 10 wt % in the electrode composition.

According to an embodiment, the electrode composition applies the COF, which is the compound represented by Formula 1 of the disclosure, as a high oxygen permeable ionomer (HOPI). This may improve gas permeability (e.g., oxygen) of the electrode or the membrane electrode (layer), and improve performance and durability of the electrode for a fuel cell. That is, a fuel cell electrode having excellent performance improvement under a high current density condition may be provided.

According to an embodiment, the catalyst is a metal active material applied to the electrode for a fuel cell. For example, the catalyst may be a platinum-based oxygen evolution reaction (OER) catalyst. More specifically, the catalyst may include one or two or more selected from the group consisting of platinum, gold, palladium, rhodium, iridium, ruthenium, osmium, alloys thereof (e.g., Pt-M), and a mixture thereof. The alloy may be Pt—Pd, Pt—Sn, Pt—Mo, Pt—Cr, Pt—W, Pt—Ru, Pt—Ru—W, Pt—Ru—Mo, Pt—Ru—Rh—Ni, Pt—Ru—Sn—W, Pt—Co, Pt—Co—Ni, Pt—Co—Fe, Pt—Co—Ir, Pt—Co—S, Pt—Co—P, Pt—Fe, Pt—Fe—Ir, Pt—Fe—S, Pt—Fe—P, Pt—Au—Co, Pt—Au—Fe, Pt—Au—Ni, Pt—Ni, Pt—Ni—Ir, Pt—Cr, Pt—Cr—Ir, or the like.

According to an embodiment, the catalyst may have an average particle diameter of 1 nm to 50 nm; 2 nm to 35 nm; 5 nm to 20 nm; or 5 nm to 10 nm.

According to an embodiment, the electrode composition may further include a fluorine-based binder (i.e., a fluorine-based ionomer). The fluorine-based binder may be present in an amount of 0.1 wt % to 10 wt % in the electrode composition. In addition, the mass ratio of the COF to the perfluorinated binder (i.e., the perfluorinated ionomer) may be 1:0.1 to 5; 1:0.5 to 4; 1:1 to 3; or 1:2 to 3. The fluorine-based binder is a perfluorinated binder (i.e., a perfluorinated ionomer). The perfluorinated binder may include a perfluorinated polymer such as poly (PFSA), a copolymer of PFSA and polytetrafluoroethylene (PTFE), poly(perfluorocarboxylic acid), a copolymer of tetrafluoroethylene containing a sulfonic acid group and fluorovinyl ether, and defluorinated sulfonated polyether ketone, but is not limited thereto.

According to an embodiment, the electrode composition may further include a solvent. The solvent may be a remaining amount or 10 wt % to 90 wt %. The solvent may be water, an organic solvent, or the like, but is not limited thereto. For example, the organic solvent may be alcohol, methanol, ethanol, isopropyl alcohol, heptanone, octanone, methoxybenzene, dimethoxypropane, N-methylpyrrolidone, dimethyl sulfoxide, tetrahydrofuran, or the like, but is not limited thereto.

According to an embodiment, the electrode composition further includes a functional additive. The functional additive may be a radical scavenger, an ionic conductor, a gas barrier particle, or the like, but is not limited thereto.

According to an embodiment, the disclosure may provide an electrode or a membrane electrode (i.e., an electrode layer of an MEA) manufactured using the electrode composition of the disclosure. The electrode is a fuel cell electrode, and may form an MEA where the electrode is applied as an electrode film disposed or coated on an electrolyte membrane. In the MEA, an electrode film manufactured using the electrode composition of the disclosure may be formed on one surface or both surfaces of the electrolyte membrane, and/or a different type of electrode film may be disposed on the other surface of the electrolyte membrane.

According to an embodiment, the electrolyte membrane may be a polymer electrolyte membrane known and sold in the technical field of the disclosure. For example, the electrolyte membrane may be in the form of a reinforced membrane in which an ion conductor fills the pores of a porous support, such as a fluorine-based porous support like e-PTFE or a porous nanoweb support manufactured by electrospinning. The ion conductor may be a cation conductor having a cation exchange group capable of transferring a cation, such as a proton, or an anion conductor having an anion exchange group capable of transferring an anion, such as a hydroxy ion, a carbonate ion, or a bicarbonate ion. The cation exchange group may be any one selected from the group consisting of a sulfonic acid group, a carboxyl group, a boronic acid group, a phosphoric acid group, an imide group, a sulfonimide group, a sulfonamide group, and a combination thereof, and generally may be a sulfonic acid group or a carboxyl group.

According to an embodiment, a method of manufacturing the electrode (film or layer) may include manufacturing an electrode composition and coating the catalyst composition on an electrolyte membrane. The manufacturing of the electrode composition may include adding a catalyst and a COF to a solvent and dispersing the catalyst and the COF to manufacture a composition suitable for a coating method. The coating may use an ultrasonic spray coating process. The coating film may be in the form of a thin film having a catalyst loading amount of 0.1 to 0.5 mg cm⁻² based on the catalyst.

According to an embodiment, the disclosure may provide a fuel cell including an electrode or an MEA manufactured using the electrode composition of the disclosure. The fuel cell may be a polymer electrolyte fuel cell. The fuel cell may further include a configuration known in the technical field of the disclosure for driving, but specific details are not mentioned herein. For example, the configuration may be a separator, a gasket, an enclosure, or the like, but is not limited thereto.

### Example 1

Synthesis of COF-S Capable of Coordination Bonding with Platinum Catalyst, Represented by Formula A

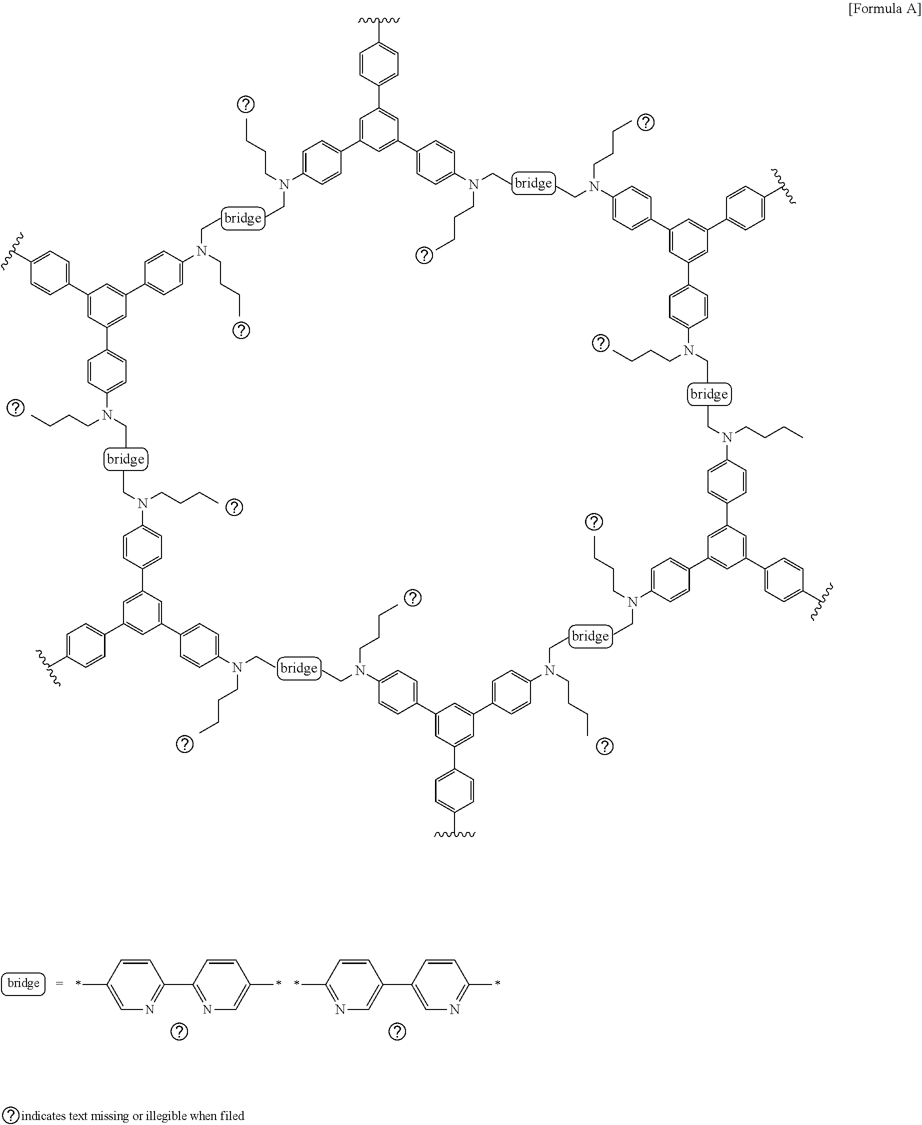

A monomer including an amine group and a monomer including an aldehyde group were dissolved in an organic solvent at a molar ratio of 1:1.5 to prepare a mixture. The mixture was placed on a plate at 120 °C, reacted for 72 hours, and then cooled to room temperature. The reacted mixture was Soxhlet washed with acetone, tetrahydrofuran, and methanol to obtain COF. The obtained COF was dried, transferred to a reaction flask, and dispersed in a tetrahydrofuran solvent. Acetic anhydride was added to the dispersion solution, and then sodium cyanoborohydride was slowly added. Thereafter, the mixture was reacted at room temperature for 24 hours, and then Soxhlet washed with acetone, tetrahydrofuran, and methanol to obtain COF-R. The obtained COF-R was dried, transferred to a reaction flask, and dispersed using dimethyl sulfoxide as a solvent. Sodium carbonate, potassium iodide, and sodium 3-bromopropanesulfonate were added to the dispersion solution. The mixture was placed on a plate at 120 °C, reacted for 48 hours, cooled to room temperature, and then washed using methanol and water. The washed product was put into a 1 M perchloric acid aqueous solution and reacted for 12 hours. The pH concentration of the reacted mixture was adjusted to a natural state. The mixture was then dried to obtain COF-S.

### Preparation of Catalyst Layer-Based MEA and Polymer Electrolyte Membrane Fuel Cell (PEMFC) Unit Cell

An MEA was prepared by a catalyst layer coating method of spray coating a catalyst ink on a Nafion 211 electrolyte membrane (DuPont®, USA). The catalyst ink was prepared as a uniform ink by mixing a commercial platinum catalyst, a 5 wt % ionomer resin solution (solvent: mixed liquid of n-propanol and distilled water; 3M, USA), isopropanol (Honeywell, Germany), and the synthesized COF-S sample, and then ultrasonically dispersing the mixture for 1 hour. The prepared ink was applied evenly using a spray on the electrolyte membrane covered with a mask having a hole with a size of 5 cm² to coat a cathode catalyst layer in the form of a thin film having a platinum-based catalyst loading amount of 0.2 mg cm⁻². On the opposite side of the electrolyte membrane, a commercial platinum catalyst ink was prepared in the same manner as above. An anode catalyst layer was then coated in the form of a thin film having a platinum-based catalyst loading amount of 0.2 mg cm⁻². Thereafter, a gas diffusion layer (GDL; 39BB, Sigracet® SGL Carbon Inc., Germany) including a microporous layer (MPL) and a gasket were fixed on upper and lower sides of the electrolyte membrane coated with the catalyst layer to prepare a 7-layer MEA. The prepared 7-layer MEA was inserted into a unit cell module composed of a graphite substrate including a serpentine-shaped gas flow channel to prepare a PEMFC unit cell.

### EXPERIMENTAL EXAMPLE

### Current-Voltage (I-V) Performance Analysis of PEMFC Unit Cell

To evaluate the current-voltage (I-V) performance of the PEMFC unit cell prepared in the Example, the PEMFC unit cell was connected to a fuel cell station (C&L Energy, Korea). The temperature of the PEMFC was set to 80 °C. The flow rate was then set so that hydrogen gas was 0.5 L min⁻¹ at an anode and air was 1.5 L min⁻¹ at a cathode. After applying a voltage of 0.4 V for 3 hours or more for activation of the PEMFC unit cell, an I-V curve was obtained using a current scanning method by setting the relative humidity of 100% and the backpressure of 0.5 bar.

### PEMFC Unit Cell Oxygen Gain Analysis

Oxygen gain analysis was performed on the prepared PEMFC unit cell. The analysis was performed under the same conditions as the ‘Current-Voltage (I-V) Performance Analysis of PEMFC Unit Cell’, but a difference in cell voltage was measured at the same current density by evaluating a gas at the cathode in two atmospheres of air and oxygen.

### Electrochemical Impedance Spectroscopy (EIS) Resistance Measurement of PEMFC Unit Cell

Resistance of the prepared 5 cm² unit cell was measured through EIS using potentiostat equipment (HCP-803, BioLogic, France). Measurement was performed from 50 kHz to 0.1 Hz in voltage regions of 0.8 V, 0.6 V, and 0.4 V using a potentiostatic mode. The flow rate was set so that hydrogen gas was 0.5 L min⁻¹ at an anode and air was 1.5 L min⁻¹ at a cathode. Measurement was then performed under conditions of a relative humidity of 100%, a cell temperature of 80 °C, and normal pressure.

### Proton Conduction Resistance Measurement

Proton resistance of the prepared 5 cm² unit cell was measured through EIS using potentiostat equipment (HCP-803, BioLogic, France). Measurement was performed in a voltage of 0.5 V and a frequency range of 20 kHz to 1 Hz. The flow rate was set so that hydrogen gas was 0.2 L min⁻¹ at the anode and nitrogen gas was 0.2 L min⁻¹ at the cathode. Measurement was then performed under conditions of a relative humidity of 100%, a cell temperature of 80 °C, and backpressure of 0.5 bar.

### Oxygen Permeation Resistance Measurement of PEMFC Unit Cell

To evaluate the oxygen permeation resistance of the prepared PEMFC unit cell through a limiting current method, the PEMFC unit cell was connected to a fuel cell station (C&L Energy, Korea). The temperature of the PEMFC was set to 80 °C. The flow rate was then set so that hydrogen gas was 1.0 L min⁻¹ at the anode and 2% O₂ was 2.0 L min⁻¹ at the cathode. A voltage was applied for 30 seconds for every 0.05 V interval from 0.95 V to 0.1 V. A limiting current density was then measured through the above method. The oxygen permeation resistance in an electrode layer was calculated using the limiting current density and the following equation:

$${R}_{t o t} = \frac{( {C}_{O 2} )}{{I}_{L} / 4 F}$$

$${C}_{02} = \frac{( P - {P}_{H 2 O} )}{R T} * {X}_{O 2}$$

I_{L}: limiting current density (A/cm²)
C_{O2}: oxygen concentration
P: absolute gas pressure (Pa)
P_{H2O}: water vapor pressure
R: gas constant (J mol⁻¹ K⁻¹)
T: gas temperature (K)
F: Faraday's constant (C mol⁻¹)

### Accelerated Degradation Test (ADT) Durability Evaluation of PEMFC Unit Cell

The ADT was performed on the 5 cm² unit cell using potentiostat equipment (HCP-803, BioLogic, France). The flow rate was set so that hydrogen gas was 0.2 L min⁻¹ at the anode and nitrogen was 0.2 L min⁻¹ at the cathode. The test was performed by a method of constantly and repeatedly applying each potential of 0.6 V and 0.95 V for 2.5 seconds each. A ramping time to each voltage was 0.5 seconds, and a total of 30,000 cycles were performed. After measuring the performance of the MEA after the ADT using the above evaluation method, I-V curves before and after the ADT were compared and evaluated.

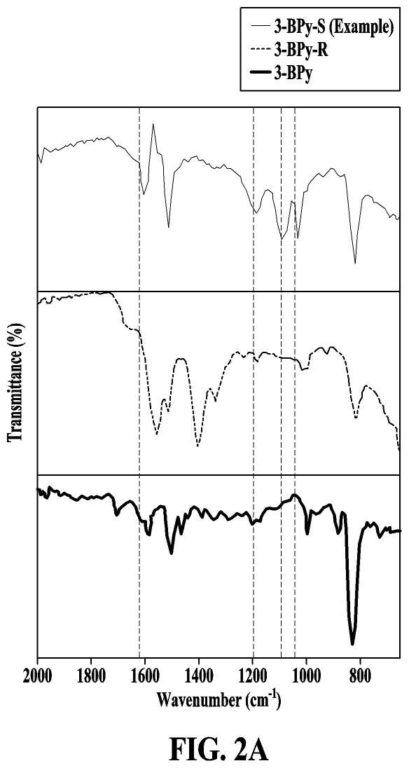

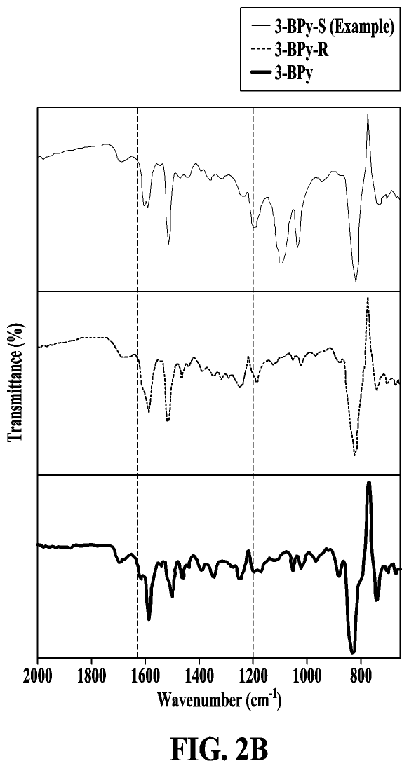

FIGS. 2A and 2B illustrate Fourier-transform infrared spectroscopy (FT-IR) spectra of COF-S prepared in Example 1, according to an embodiment.

From the FT-IR analysis shown in FIGS. 2A and 2B, it was confirmed that COF-S was synthesized to have the structure of Formula 1. It was confirmed that peaks related to an imine group of COF-S at 1620.98 cm⁻¹ and 1624.10 cm⁻¹ disappeared, and peaks related to an amine group of COF-R appeared at 1201.27 cm⁻¹ and 1186.69 cm⁻¹. Peaks related to a sulfonic acid group of COF-S may be confirmed at 1094.2 cm⁻¹, 1036.7 cm⁻¹ (2-BPy-S), 1096.1 cm⁻¹, and 1036.2 cm⁻¹ (3-BPy-S).

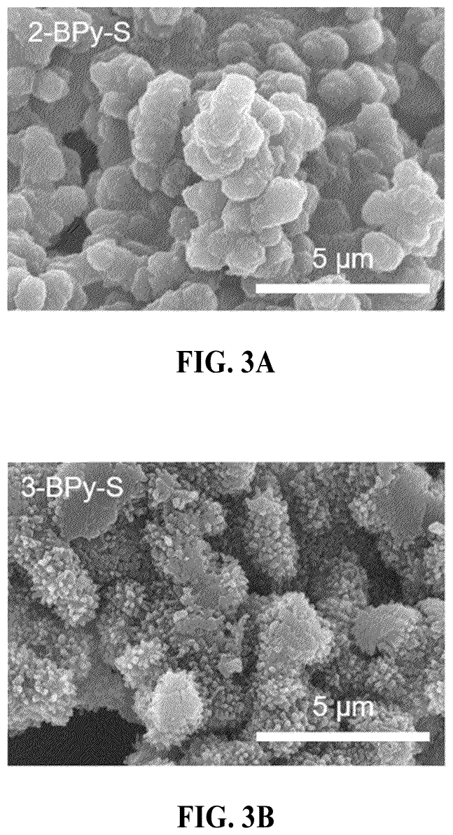

FIGS. 3A and 3B show scanning electron microscope (SEM) photographs of COF-S capable of coordination bonding with a platinum catalyst prepared in Example 1, according to an embodiment.

From the SEM analysis shown in FIGS. 3A and 3B, it was confirmed that 2-BPy-S and 3-BPy-S were synthesized as COF-S having a spherical shape with particle sizes of less than about 5 μm.

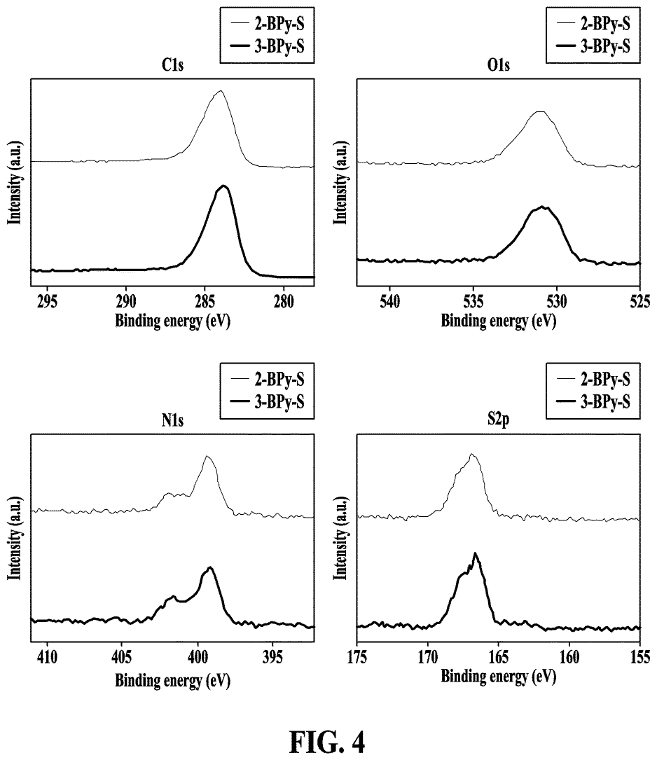

FIG. 4 illustrates an X-ray photoelectron spectroscopy (XPS) spectrum of COF-S capable of coordination bonding with a platinum catalyst synthesized in Example 1. From the XPS analysis shown in FIG. 4, it was confirmed that COF-S was synthesized to have the structure of Formula 1.

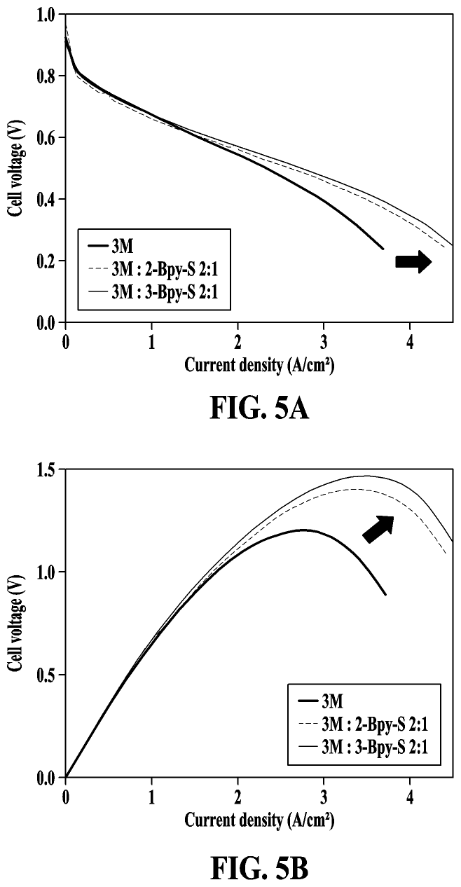

FIGS. 5A and 5B illustrate current-voltage performance (H₂/Air) of a PEMFC unit cell prepared in Example 1, according to an embodiment.

As shown in FIGS. 5A and 5B, the PEMFC single cell of Example 1 secured a higher voltage at the same current density compared to a single cell based on a 3M PFSA ionomer.

In addition, the PEMFC unit cell of Example 1 shows a higher power density than a commercial platinum catalyst-based unit cell, indicating a performance improvement effect due to the introduction of a COF-S material. That is, it may be confirmed that in the case of an MEA using only a 3M ionomer, a maximum power density was 1.202 W/cm², whereas in the case of an MEA to which COF-S (Example 1) is applied, a maximum power density was 1.467 W/cm².

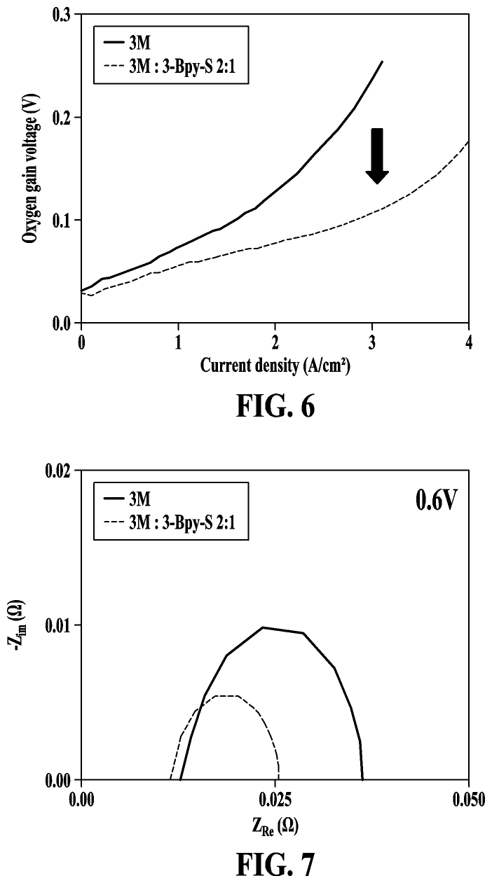

FIG. 6 illustrates an oxygen gain analysis of a PEMFC unit cell prepared in Example 1, according to an embodiment. FIG. 6 was calculated through current-voltage curves evaluated in H₂/Air and H₂/O₂ atmospheres, respectively. A difference in cell voltage was then measured at the same current density.

An oxygen gain voltage of a unit cell to which COF-S was added was measured to be low, which means that the mass transport resistance of the unit cell MEA to which COF-S (Example 1) was added was lowered, enabling smooth gas transport in an electrode.

FIG. 7 illustrates EIS resistance measurement of a PEMFC unit cell prepared in Example 1, according to an embodiment.

As shown in FIG. 7, it was confirmed that the resistance of a material to which COF-S (Example 1) was added was lower than that of an MEA using a PFSA-based ionomer, which is presumed to be due to improved mass transport efficiency.

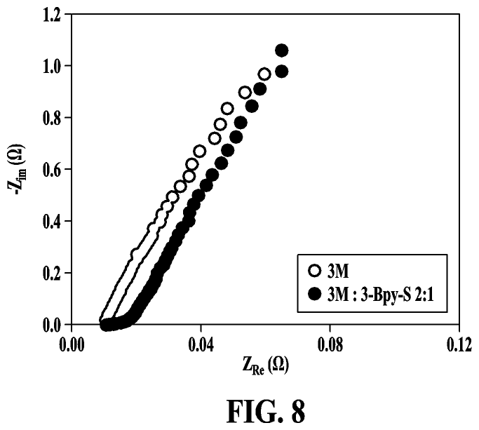

FIG. 8 illustrates resistance measured through EIS of an MEA (3M) using only a PFSA-based ionomer and a sample to which a COF-S material of Example 1 is added, according to an embodiment.

This represents proton resistance measured through EIS of the MEA (3M) using only a PFSA-based ionomer and the sample to which the COF-S material is added. In FIG. 8, in the case of the MEA to which the COF-S material is added, it may be confirmed that resistance to proton conduction is higher than that of the MEA using a PFSA-based ionomer because the MEA to which the COF-S material is added has a problem in that it is difficult to form a large ion transfer channel like a perfluorinated ionomer.

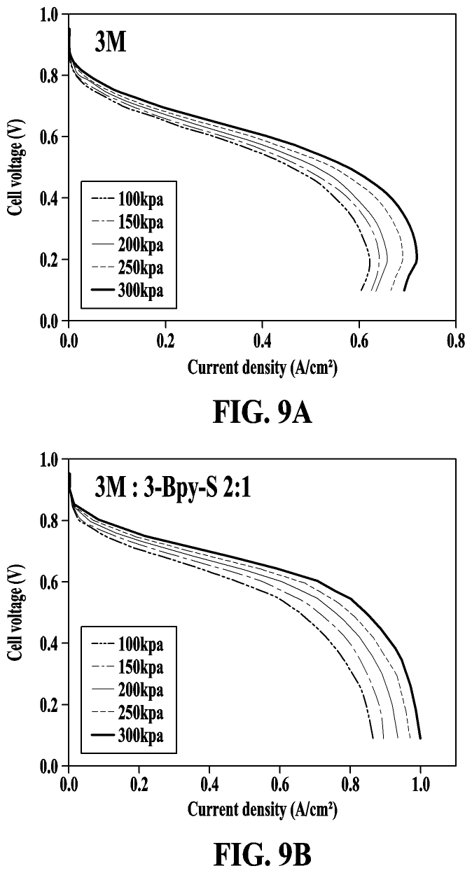

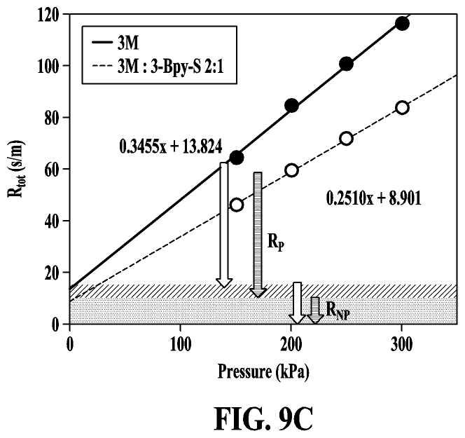

FIGS. 9A, 9B, and 9C illustrate oxygen permeation resistance measurement of a PEMFC unit cell prepared in Example 1, according to an embodiment.

That is, limiting current densities of the MEA using only a PFSA-based ionomer and the MEA to which the COF-S material (Example 1) is added were measured. It may be confirmed that the limiting current density of the MEA to which the COF-S material is added is higher than that of the MEA using only a PFSA-based ionomer, and this is because transfer of a reactant occurs smoothly. In addition, it may be confirmed that resistance to oxygen permeation calculated through the limiting current density was also measured to be low in the sample into which the COF-S material is put.

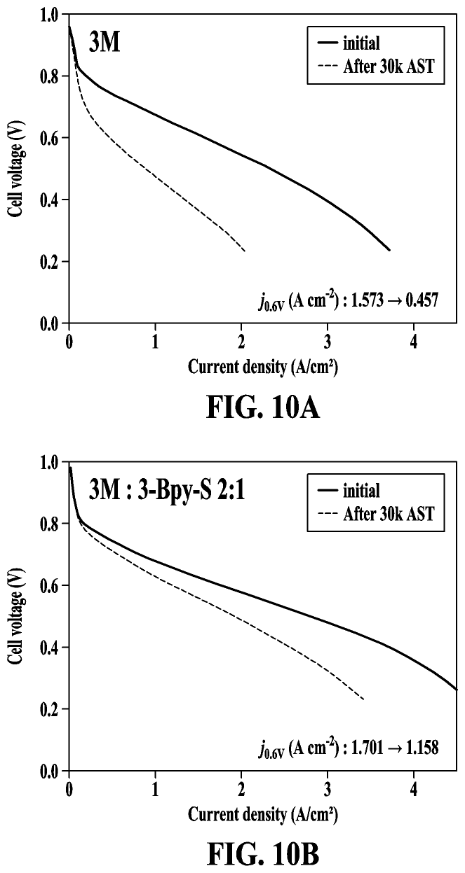

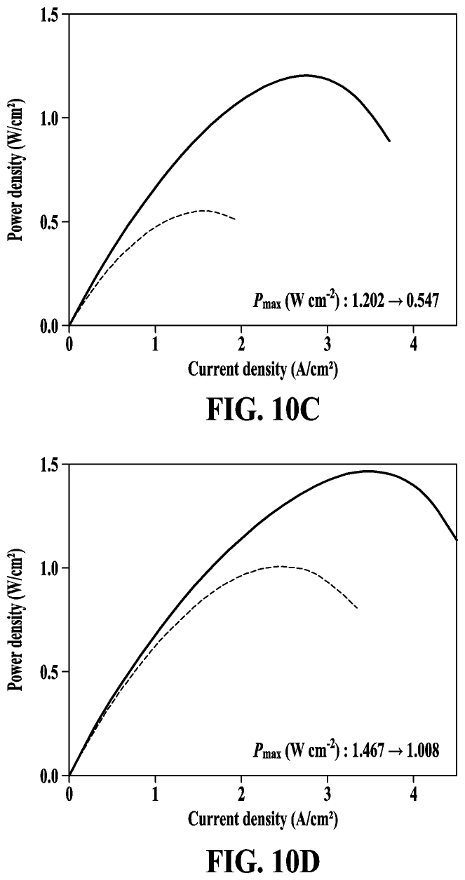

FIGS. 10A, 10B, 10C and 10D illustrate current-voltage performance of a PEMFC unit cell prepared in Example 1 before and after an ADT, according to an embodiment.

That is, current-voltage curves before and after 30,000 cycles of the ADT of the MEA (3M) using only a PFSA-based ionomer and the MEA (3M: 3-Bpy 2:1) sample to which the COF-S material (Example 1) is added are compared. It may be confirmed that a retention rate of a maximum current density of the MEA to which the COF-S material is added is higher than that of the MEA using only a PFSA-based ionomer. This is because nitrogen included in the COF-S material binds platinum particles, thereby preventing or reducing loss of platinum.

The disclosure may provide a COF capable of coordination bonding with a platinum catalyst and having a sulfonic acid group, which is utilized as a novel polymer electrolyte fuel cell binder. The disclosure may also provide a COF-based polymer electrolyte membrane electrode layer of an MEA, a fuel cell electrode, and a fuel cell. That is, the COF of the disclosure enables coordination bonding with a platinum catalyst applicable to MEA driving in a driving environment of a PEMFC, enables high oxygen permeation, and may ensure durability for the platinum catalyst.

A number of embodiments have been described above. Nevertheless, it should be understood that various modifications may be made to these embodiments. For example, suitable results may be achieved if the described techniques are performed in a different order and/or if the described components are combined in a different manner and/or replaced or substituted by other components or their equivalents. Therefore, other implementations, other embodiments, and equivalents to the claims are also within the scope of the following claims.

What is claimed is:

1. A covalent organic framework (COF), which is a compound represented by Formula 1: 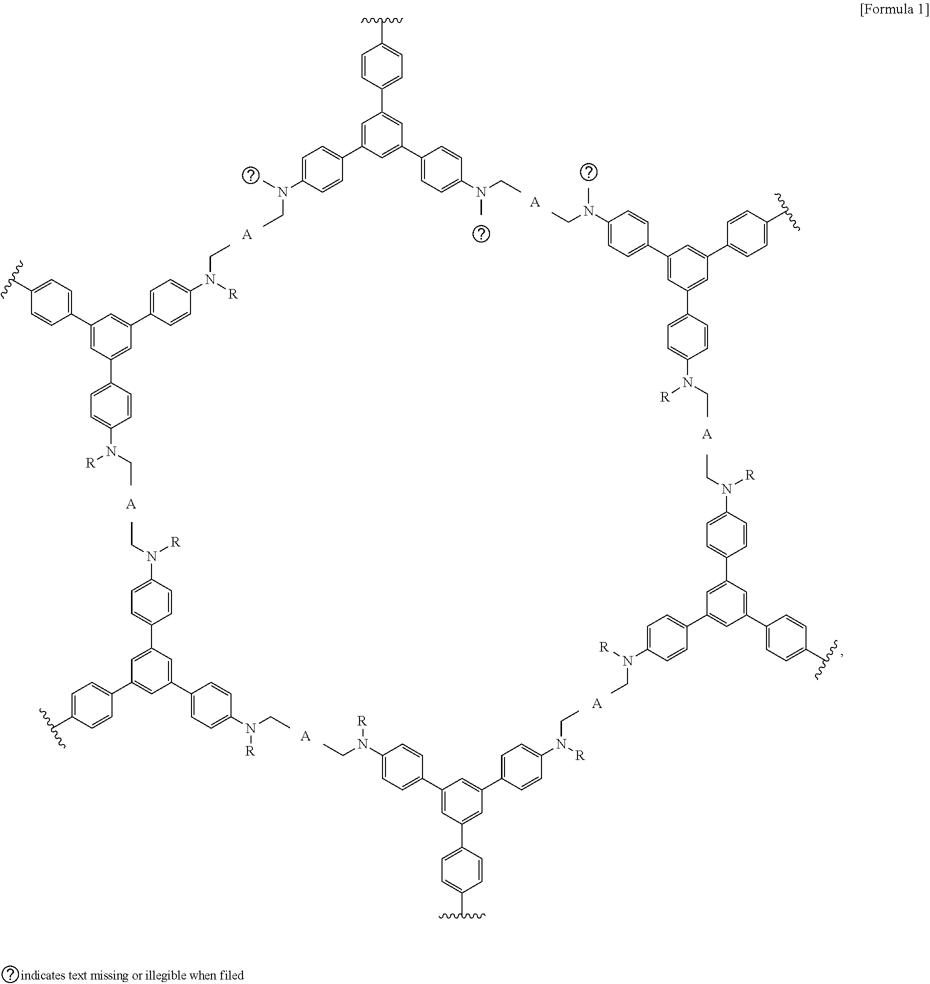
wherein R is an ionic group and is —(CH₂)n-Z⁻, n is 1 to 10, and Z⁻ is COO⁻, SO₃⁻, or PF₅⁻, and
A is a bridging group in which two or more six-membered aromatic rings, each containing one or more nitrogen atoms in a ring, are connected.

2. The COF of claim 1, wherein, in Formula 1, Z⁻ is SO₃⁻; and n is 3 to 4.

3. The COF of claim 1, wherein A is represented by the following: 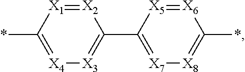
wherein at least two of X₁ to X₈ are nitrogen, and * denotes a connection site.

4. The COF of claim 1, wherein A is selected from the following:

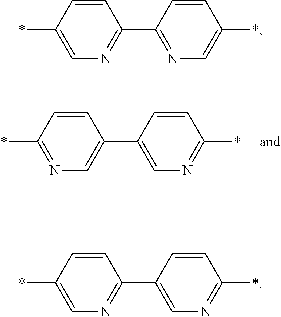

5. An electrode composition for a fuel cell, the electrode composition comprising:
a covalent organic framework (COF) that is a compound represented by Formula 1 of claim 1; and
a catalyst for a fuel cell.

6. The electrode composition of claim 5, wherein the COF is present in an amount of 0.1 weight percent (wt %) to 10 wt % in the electrode composition.

7. The electrode composition of claim 5, wherein a bridging group in a molecule of the COF and the catalyst form a coordination bond.

8. The electrode composition of claim 5, further comprising a perfluorinated binder, wherein a mass ratio of the COF to the perfluorinated binder is 1:0.1 to 5.

9. The electrode composition of claim 8, wherein the perfluorinated binder is Nafion.

10. The electrode composition of claim 5, wherein the catalyst comprises at least one selected from the group consisting of platinum, gold, palladium, rhodium, iridium, ruthenium, osmium, and alloys thereof.

11. A membrane-electrode assembly (MEA) comprising:
an electrolyte membrane; and
an electrode layer comprising the electrode composition for a fuel cell of claim 5, wherein the electrode layer is disposed on one surface or both surfaces of the electrolyte membrane.
